(19) United States
(12) Patent Application Publication
(10) Pub. No.: US 2022/0202673 A1
(43) Pub. Date: Jun. 30, 2022

(54) Silicone-Free Personal Care Compositions and Methods for the Same

(71) Applicant: Colgate-Palmolive Company, New York, NY (US)

(72) Inventors: Esinam Kuma AGBLEY, Somerset, NJ (US); Arturo ZUNIGA, Ciudad de México (MX); Edgar H. HERNANDEZ, Residencial Acueducto de Guadalupe (MX)

(73) Assignee: Colgate-Palmolive Company, New York, NY (US)

(21) Appl. No.: 17/563,293

(22) Filed: Dec. 28, 2021

Related U.S. Application Data
(60) Provisional application No. 63/132,431, filed on Dec. 30, 2020.

(51) Int. Cl. A61K 8/42; A61Q 5/12; A61K 8/06; A61K 8/365; A61K 8/368; A61K 8/86; A61K 8/34

(52) U.S. Cl. CPC A61K 8/42; A61Q 5/12; A61K 8/062; A61K 2800/34; A61K 8/368; A61K 8/86; A61K 8/342; A61K 8/365

(57) ABSTRACT

Silicone-free personal care compositions and methods for the same are disclosed. The personal care composition may include an emulsifying system having one or more surfactants, one or more emollients, and one or more conditioning agents. The personal care composition may include a natural origin index of greater than 95%.

### BACKGROUND

Conventional hair care products often incorporate conditioning agents to deliver or provide benefits to hair, thereby improving consumer perception of the hair care product. For example, smoothening or softening hair surface or surfaces of hair is a fundamental benefit of the conditioning agents that improves consumer perception, as softening surfaces of the hair eases wet and dry combing, provides a soft feel, improves shine, and reduces frizz and tangling. The most common conditioning agents include silicon-based conditioning agents. Silicone, however, is a non-natural ingredient and recent consumer awareness and perception regarding non-natural ingredients have motivated the development of more natural compositions or formulations via the inclusion of more naturally derived ingredients.

In view of the foregoing, efforts have been directed to reducing or eliminating non-natural ingredients, such as petrochemical emollients, mineral oils, synthetic polymers, and/or silicone, from personal care compositions to provide improved consumer perception. For example, many personal care manufacturers are now providing silicon-free shampoos, conditioners, or other products to consumers. These silicone-free products, however, do not deliver hair conditioning benefits comparable to the silicone-based compositions and many consumers do not want to compromise the conditioning and softening benefits of the silicon-based products for more natural products.

What is needed, then, are improved silicone-free personal care compositions having hair conditioning benefits comparable to silicone-based compositions.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing a personal care composition. The personal care composition may include an emulsifying system having one or more surfactants, one or more emollients, and one or more conditioning agents. The personal care composition may include a natural origin index of greater than 95%.

In at least one implementation, the personal care composition may be substantially free of one or more of silicon, silicon compounds, cetrimonium chloride, propylene glycol, mineral oil, petrolatum, polyethylene glycol, or combinations thereof.

In at least one implementation, the personal care composition may be substantially free of silicon and silicon compounds.

In at least one implementation, the personal care composition may be substantially free of silicon, silicon compounds, cetrimonium chloride, propylene glycol, mineral oil, petrolatum, and polyethylene glycol.

In at least one implementation, the conditioning agents may include a cationic conditioning agent, preferably the cationic conditioning agent may include brassicamidopropyl dimethylamine.

In at least one implementation, the conditioning agent may be present in an amount of from 0.1 weight % to about 5 weight %, based on the total weight of the personal care composition.

In at least one implementation, the one or more surfactants may include one or more nonionic surfactants, preferably the nonionic surfactants may include one or more of cetyl alcohol, stearyl alcohol, or combinations thereof.

In at least one implementation, the nonionic surfactants may include a combination of cetyl alcohol and stearyl alcohol.

In at least one implementation, the one or more surfactants may be present in an amount of from greater than 0 weight % to about 20 weight %, preferably from 3 weight % to about 7 weight %, more preferably about 4 weight % to about 6 weight %, based on the total weight of the personal care composition.

In at least one implementation, the one or more emollients may include a polypropylene glycol alkyl ether, preferably the polypropylene glycol alkyl ether comprises PPG-3 Caprylyl ether.

In at least one implementation, the emollients may be present in an amount of from greater than 0 weight % to about 5 weight %, more preferably, about 0.1 weight % to about 0.5 weight %, more preferably from about 0.1 weight % to about 0.3 weight %.

In at least one implementation, wherein the natural origin index may be greater than 96%, more preferably greater than 98%.

In at least one implementation, the personal care composition may further include a preservative, optionally the preservative may include one or more of sodium benzoate, potassium sorbate, lactic acid, or combinations thereof, further optionally, the preservative may include a combination of sodium benzoate, potassium sorbate, and lactic acid.

In at least one implementation, the personal care composition may exhibit substantially similar or greater properties as compared to a conventional personal care composition including any one or more of silicone, silicon compounds, cetrimonium chloride, propylene glycol, mineral oil, petrolatum, polyethylene glycol, or combinations thereof.

The foregoing and/or other aspects and utilities embodied in the present disclosure may be achieved by providing a method for delivering conditioning benefits to hair of a mammal, the method may include contacting any one of the personal care compositions disclosed herein with the hair of the mammal in need thereof. The conditioning benefits may include one or more of facilitating wet combing, facilitating dry combing, softness, shine, reducing hair fly-aways, improving hair alignment, facilitating detangling of hair, or combinations thereof.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight (wt. %). The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that cetrimonium chloride and silicon of personal care compositions may be replaced with brassicamidopropyl dimethylamine and PPG-3 caprylyl ether, respectively, to provide a silicon-free personal care composition having conditioning benefits comparable to silicon-based personal care compositions.

Compositions disclosed herein may be or include a personal care product or a personal care composition thereof. The personal care composition may be a leave-in or leave-on composition (e.g., leave-in or leave-on hair conditioner) or a rinse-off composition (e.g., rinse-off hair conditioner). The personal care composition may be or include a hair care product or a hair care composition thereof. Illustrative hair care products may be or include, but are not limited to, shampoos, leave-in or rinse-off conditioners, leave-in or rinse-off deep treatments, hair spray, gels, or the like. In an exemplary implementation, the composition is a leave-in or a rinse-off conditioner.

The compositions disclosed herein may have a natural origin index of greater than about 90%. As used herein, the term or expression “natural origin index” or “natural origin content” may refer to a value (e.g., a percentage) or degree of a substance (e.g., a product or a composition thereof) that describes the natural content of ingredients or formulation of the substance. For example, the natural origin index may be a mass percentage between 0% and 100% of all natural ingredients and natural portions of derived natural ingredients in a product. The natural origin index may be determined by the ISO 16128-2:2017 standard of the International Organization for Standardization (ISO). In at least one example, the natural origin index of the personal care compositions disclosed herein may be greater than 90%, greater than 95%, greater than 96%, greater than 97%, greater than 98%, greater than 99%, greater than 99.5%, or 100%.

The compositions disclosed herein may include an emulsifying system including one or more surfactants or emulsifiers, one or more emollients, one or more conditioning agents, or any combination thereof. The emulsifying system, the one or more emollients, and/or the one or more conditioning agents may be disposed, dispersed, or otherwise contained in a vehicle or carrier. The compositions disclosed herein may be free or substantially free of silicone, silicone compounds, propylene glycol, mineral oil, petrolatum, polyethylene glycol (PEG), petrochemical emollients, synthetic polymers, or any combination thereof. For example, the composition may include silicone or silicon compounds and be free or substantially free of PEG. In another example, the composition may include PEG and be free or substantially free of silicone, silicone compounds, propylene glycol, mineral oil, petrolatum, petrochemical emollients, synthetic polymers, or any combination thereof. In another example, the personal care composition may be free or substantially free of silicone, silicone compounds, propylene glycol, mineral oil, petrolatum, and polyethylene glycol (PEG). The personal care compositions may also be free or substantially free of cetrimonium chloride. Illustrative silicone or silicone compounds may be or include, but are not limited to, polysiloxane polymers, such as dimethicone, dimethiconol, cyclopentasiloxane, modified silicones, or any combination thereof.

The personal care compositions disclosed herein may exhibit comparable, enhanced, improved, or relatively greater (e.g., statistically significantly greater) properties and/or features as compared to conventional personal care compositions including silicone, silicone compounds, cetrimonium chloride, propylene glycol, mineral oil, petrolatum, polyethylene glycol (PEG), or any combination thereof. For example, the personal care compositions disclosed herein may exhibit substantially similar or relatively greater performance with respect to any one or more of the following: ease of application in wet hair, ease of distribution in wet hair, ease of finger detangling while rinsing, ease of detangling wet hair, ease of combing wet hair, ease of combing dry hair, softness of dry hair, softness, shine, fly-aways, alignment, or any combination thereof, as compared to conventional personal care compositions including any one or more of silicone, silicone compounds, cetrimonium chloride, propylene glycol, mineral oil, petrolatum, polyethylene glycol (PEG), or any combination thereof. As used herein, the term or expression “substantially similar” may refer to a property or value of a test composition that does not change (e.g., increase or decrease) from a comparative composition by an amount greater than 0.5%, greater than 1%, greater than 1.5%, greater than 2%, greater than 2.5%, greater than 3%, greater than 3.5%, greater than 4%, greater than 4.5%, or greater than 5%. In at least one implementation, the personal care composition may include an emulsifying system, one or more emollients, one or more conditioning agents, or any combination thereof capable of or configured to provide comparable (e.g., substantially similar) or relatively greater properties and/or features to the personal care composition as compared to conventional personal care compositions including any one or more of silicone, silicone compounds, cetrimonium chloride, propylene glycol, mineral oil, petrolatum, polyethylene glycol (PEG), or any combination thereof.

As used herein, the term or expression “personal care composition” may refer to a composition for topical application to the skin of mammals, especially humans. The personal care composition may generally be a leave-on personal care composition or rinse off personal care composition, and may include any product applied to a human body. The personal care composition is preferably a leave-on personal care composition. The personal care composition may be in any suitable form. Illustrative forms of the personal care composition may be or include, but is not limited to, a liquid, a lotion, a cream, a foam, a scrub, a gel, a solid, a soap bar, a toner, applied with an implement or via a face mask, or the like. Illustrative personal care compositions may be or include, but are not limited to, leave-on skin lotions and creams, emulsion, shampoos, conditioners, shower gels, antiperspirants, deodorants, depilatories, lipsticks, foundations, mascara, sunless tanners, sunscreen lotions, or the like.

The personal care composition may have an acidic to neutral pH. For example, the personal care composition may have a pH of from about 3, about 3.5, about 4, or about 4.5 to about 5, about 5.5, about 6, about 6.5, or about 7. In another example, the personal care composition may have a pH of less than or equal to 7, less than 6.5, less than 6, less than 5.5, less than 5, or less than 4.5. In a preferred implementation, the personal care composition may have a pH of from about 4.0 to about 5.0, or about 4.2 to about 4.8, more preferably about 4.2 to about 4.6.

The personal care composition may have a viscosity, as determined using a viscometer at a temperature of about 25° C., of greater than or equal to about 7,000 centipoise (cP), greater than about 8,000 cP, greater than about 10,000 cP, greater than about 12,000 cP, greater than about 13,000 cP, greater than about 14,000 cP, or greater than about 15,000 cP. As used herein, the term “viscosity” may refer to the internal resistance to flow exhibited by a fluid or the ratio of shearing stress to rate of shear, and may be measured in poise or centipoises (cP).

As previously disclosed, the emulsifying system, the emollients, and/or the conditioning agents may be dispersed or otherwise contained in a vehicle or carrier. The vehicle or carrier may be or include a cosmetically acceptable solvent. Illustrative vehicles or solvents thereof may be or include, but are not limited to, water (e.g., demineralized water), one or more organic solvents, or mixtures thereof. In a preferred implementation, the vehicle or carrier of the personal care composition includes water. The water may be deionized water, demineralized water, and/or softened water.

The vehicle or carrier may be present in an amount of up to about 85%, up to about 90%, up to about 91%, up to about 92%, up to 93%, up to 94%, up to about 95%, or more, based on the total weight of the personal care composition. For example, the personal care composition may include water in an amount of from about 85% to about 95%, about 90% to about 94%, about 91% to about 92%, or about 91.5%, based on the total weight of the personal care composition. Water may make up the balance of the personal care composition. For example, the amount of water present in the personal care composition may be greater than 60 weight %, greater than 65 weight %, greater than 70 weight %, greater than 75 weight %, greater than 80 weight %, greater than 85 weight %, greater than 90 weight %, greater than 92 weight %, greater than 94 weight %, greater than 96 weight %, or more, based on the total weight of the personal care composition. The amount of water in the personal care composition may include free water added and water introduced with other components or materials of the personal care composition. For example, the amount of the water in the personal care composition may include free water and water associated with one or more surfactants and/or any other components of the personal care composition.

The one or more conditioning agents of the personal care composition may be or include, but are not limited to, one or more cationic conditioning agents, anionic conditioning agents, non-ionic conditioning agents, amphoteric conditioning agents, or combinations thereof. Illustrative cationic conditioning agents may be or include, but are not limited to, polyquaterium-10 (i.e., quaternized polyhydroxyethyl cellulose), behentrimonium chloride, behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, Polyquaternium-4, Polyquaternium-6, Polyquaternium-11, Polyquaternium-44, Polyquaternium-67, amodimethicone, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, aminopropyl dimethicone, Quaterium-8, dilinoleamidopropyl dimethylamine dimethicone PEG-7 phosphate, or combinations thereof. In a preferred implementation, the one or more conditioning agents include at least one cationic conditioning agent, and the at least one cationic conditioning agent preferably includes brassicamidopropyl dimethylamine.

The one or more conditioning agents may be present in an amount of from about 0.1 wt % to about 5 wt %, based on the total weight of the personal care composition. For example, the conditioning agents may be present in an amount of from about 0.1 wt % to about 0.2 wt %, about 0.25 wt %, about 1 wt %, about 2 wt %, about 2.5 wt %, about 3 wt %, about 3.5 wt %, about 4 wt %, about 4.5 wt %, or about 5 wt %, based on the total weight of the personal care composition. In another example, the conditioning agents may be present in an amount of from about 0.1 wt %, about 0.2 wt %, about 0.25 wt %, about 1 wt %, about 2 wt %, about 2.5 wt %, about 3 wt %, about 3.5 wt %, about 4 wt %, or about 4.5 wt %, to about 5 wt %, based on the total weight of the personal care composition. In yet another example, the conditioning agents may be present in an amount of from about 0.1 wt % to about 1 wt %, about 0.2 wt % to about 0.5 wt %, or about 0.25 wt % to about 0.4 wt %.

In at least one implementation, the personal care composition may include one or more preservatives in an amount of greater than 0 weight % and less than or equal to about 3 weight %, less than or equal to about 2.5 weight %, less than or equal to about 2 weight %, less than or equal to about 1.5 weight %, less than or equal to about 1 weight %, less than or equal to about 0.8 weight %, less than or equal to about 0.7 weight %, less than or equal to about 0.25 weight %, less than or equal to about 0.1 wt %, less than or equal to about 0.08 wt %, or less than or equal to about 0.05 wt %, based on the total weight of the personal care composition. Illustrative preservatives may include, but are not limited to, benzalkonium chloride; benzethonium chloride, 5-bromo-5-nitro-1,3-dioxane; 2-bromo-2-nitropropane-1,3-diol; alkyl trimethyl ammonium bromide; N-(hydroxymethyl)-N-(1,3-dihydroxy methyl-2,5-dioxo-4-imidaxolidinyl-N-(hydroxy methyl)urea; 1-3-dimethyol-5,5-dimethyl hydantoin; formaldehyde; iodopropynl butyl carbamate, butyl paraben; ethyl paraben; methyl paraben; propyl paraben, mixture of methyl isothiazolinone/methyl-chloroisothiazoline in a 1:3 wt. ratio; mixture of polyvinylpyrro/butyl paraben/methyl paraben/propylparaben; 2-phenoxyethanol; tris-hydroxyethyl-hexahydrotriaz-ine; methylisothiazolinone; 5-chloro-2-methyl-4-isothiazolin-3-one; 1,2-dibromo-2,4-dicyanobutane; 1-(3-chloroalkyl)-3,5,7-triaza-azoniaadam-antane chloride; sodium benzoate; potassium sorbate, organic acids, sorbic acid, lactic acid, citric acid, potassium sorbate, or the like, or any combination thereof. In a preferred implementation, the one or more preservatives include sodium benzoate, potassium sorbate, lactic acid, or a combination thereof.

In at least one implementation, the one or more preservatives may be present in an amount of from greater than 0 weight % to less than or equal to 2 weight %, less than or equal to 1 wt %, less than or equal to 1.5 wt %, less than or equal to 1 wt %, less than or equal to 0.8 wt %, less than or equal to 0.5 wt %, or less than or equal to 0.3 wt %, less than or equal to about 0.25 weight %, less than or equal to about 0.1 wt %, less than or equal to about 0.08 wt %, less than or equal to about 0.06 wt %, or less than or equal to about 0.05 wt %, based on the total weight of the personal care composition. In a preferred implementation, the one or more preservatives includes sodium benzoate and lactic acid in an amount of from greater than 0 wt % to less than 0.5 weight %, more preferably less than 0.1 wt %, based on the total weight of the personal care composition.

The personal care composition may include an emulsifying system including one or more surfactants. The one or more surfactants may include, but are not limited to, one or more anionic surfactants, one or more cationic surfactants, one or more zwitterionic surfactants, one or more nonionic surfactants, or mixtures thereof.

Illustrative anionic surfactants may include, but are not limited to, stearic acid, water-soluble salts of higher fatty acid monoglyceride monosulfates, such as a sodium salt of a monosulfated monoglyceride of hydrogenated coconut oil fatty acids, such as sodium N-methyl N-cocoyl polyviny, sodium cocomonoglyceride sulfate. Illustrative anionic surfactants may also include higher alkyl sulfates. As used herein, “higher alkyl” refers to C₆₋₃₀ alkyls. For example, the anionic surfactant may be or include sodium lauryl sulfate. The anionic surfactants may also include higher alkyl-ether sulfates. In another implementation, the anionic surfactant may include higher alkyl aryl sulfonates, such as sodium dodecyl benzene sulfonate (sodium lauryl benzene sulfonate), and higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. In an exemplary implementation, the anionic surfactant may be or include a water soluble salt of alkyl sulfates having from 10 to 18 carbon atoms in the alkyl radical and water soluble salts of sulfonated monoglycerides of fatty acids having from 10 to 18 carbon atoms.

Illustrative cationic surfactants may be or include, but are not limited to, ammonium salts such as distearyldimethylammonium chloride, halide salts of methylated ammonium wherein at least one and preferably two or three of the groups on the nitrogen are long chain substantially saturated material such as tallow fatty radicals, hydrogenated tallow fatty radicals, methosulfate anionic salts of the above, long chain amidoderivative such as mink oil amidopropyldimethyl-2-hydroxyethylammonium chloride (Quaternium 26), aromatics such as isododecylbenzyl triethanol ammonium chloride, N-alkylated amphoteric materials such as N-alkylated betaines, alkylamines, alkylimidazolines, various ethoxylated amines, Quaternium-8, -14, -18, -24, -26, -27, -33, -43, -52, -53, -60, -62, -83, esterquats, or the like, or any combination thereof. Illustrative esterquats may be or include, but are not limited to, dibehenoylethyl dimonium chloride, dipalmitoylethyl dimonium chloride, distearoylethyl dimonium chloride, ditallowoyl pg-dimonium chloride, dipalmitoylethyl hydroxyethylmonium methosulfate, distearoylethyl hydroxyethylmonium methosulfate, or the like, or combinations and mixtures thereof.

Illustrative nonionic surfactants may be or include, but are not limited to, higher alcohols or fatty alcohols, including straight chain alcohols or ethoxylates thereof, sorbitan fatty acid esters (e.g., sorbitan mono oleate, sorbitan mono isostearate, sorbitan mono laurate, sorbitan mono palmitate, sorbitan mono stearate, sorbitan sesquioleate, sorbitan trioleate, diglycerol sorbitan penta-2-ethylhexylate, diglycerol sorbitan tetra-2-ethylhexylate, etc.); glycerin polyglycerin aliphatic acids (e.g., mono cottonseed oil fatty acid glycerin, glyceryl monoerucate, glycerin sesquioleate, glyceryl monostearate, glyceryl stearate, α, α′-glycerin oleate pyroglutamate, monostearate glycerin malic acid, etc.); propylene glycol fatty acid esters (e.g., propylene glycol monostearatem, etc.); hydrogenated castor oil derivatives; glycerin alkylethers, or the like, or combinations thereof. Illustrative nonionic surfactants may also be or include, but are not limited to, sorbitan esters and ethoxylated sorbitan esters (e.g., PEG-20 sorbitan isostearate, sorbitan monolaurate, polysorbate-20, polysorbate-40, polysorbate-60, polysorbate-80); ethoxylates (e.g., Ceteth-20, PEG-30 castor oil, PEG-40 hydrogenated castor oil, PEG-60 hydrogenated castor oil, Laureth-7, Isolaureth-6, Steareth-10, Steareth-20, Steareth-21, Steareth-100, Ceteareth-12, Oleth-5, Oleth-10, etc.); ethoxylated adducts (e.g., PEG-25 stearate, glyceryl stearate, PEG-100 stearate, etc.); polyoxyethylene (100) monostearate, a polyethylene glycol ester of stearic acid, PEG esters (e.g., PEG-8 oleate, PEG-8 laurate, PEG-8 dilaurate, PEG-12 dilaurate, PEG-80 diisostearate, PEG-40 stearate, etc.); propoxylates (e.g., PPG-10 butanediol, PPG-50 oleyl ether, PPG-2-ceteareth-9, PPG-3-deceth-3, PPG-5-ceteth-20); ethoxylated modified triglycerides (e.g., PEG-20 corn glycerides, PEG-12 palm kernel glycerides); alkylphenol aromatic ethoxylates (e.g., dinonylphenol ethoxylate with 9 moles of EO octylphenol ethoxylate with 20 moles of EO, octylphenol ethoxylate with 40 moles of EO); block copolymers that are alkoxylated glycols having ethoxylated and propoxylated segments (e.g., POLOXAMER™ 182 and 234, POLOXAMER™ 105 Benzoate, and MEROXAPOL™ 174); or the like, or combinations thereof. Illustrative ethoxylates may be or include, but are not limited to, Ceteth-20, PEG-30 castor oil, PEG-40 hydrogenated castor oil, PEG-60 hydrogenated castor oil, Laureth-7, Isolaureth-6, Steareth-10, Steareth-20, Steareth-21, Steareth-100, Ceteareth-12, Oleth-5, Oleth-10, or combinations thereof.

Illustrative nonionic surfactants may also include, but are not limited to, the reaction products of aliphatic alcohols or alkylphenols having 6 to 20 carbon atoms in a linear or branched alkyl chain with ethylene oxide and/or propylene oxide where the alkylene oxide may be from about 6 moles to about 60 moles per mole of alcohol. For example, the nonionic surfactants may include, but are not limited to, alkylamine oxides, mono- and dialkylalkanolamides, fatty acid esters of polyethylenenglycols, ethoxylated fatty acids amides, saturated fatty acid alcohols reacted with ethylene oxide, alkyl polyglycosides, and sorbitan ether esters, one or more Ceteareth-n, such as Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, or the like, or combinations thereof, or one or more Ceteareth in combination with a fatty acid alcohol such as stearyl alcohol, oleyl alcohol, linoleyl alcohol, arachidyl alcohol, cetyl alcohol, cetearyl alcohol (i.e., a mixture of cetyl and stearyl alcohols), or the like, or any combination thereof. It should be appreciated that Ceteareth-n is a name of an INCI family and refers to polyoxyethylene ethers of a mixture of high molecular mass saturated fatty alcohols, where the ‘n’ indicates the average number of ethylene oxide residues in the polyoxyethylene chain. In an exemplary implementation, the nonionic surfactants include cetearyl alcohol, ceteareth-20, cetyl alcohol, stearyl alcohol, or combinations thereof. It should be appreciated that Ceteareth-20 (CAS Number 68439-49-6) is a polyethylene glycol ether of cetearyl alcohol.

In a preferred implementation, the one or more nonionic surfactants include one or more polypropylene glycol alkyl ethers according to structure (1):

CₙH₂ₙ₊₁(OCH₂(CH₃)CH₂)ₓOH (1)

where “n” is an integer of from about 1 to about 12, and “x” is an integer of from about 1 to about 40, preferably about 3 to about 30. Illustrative polypropylene glycol alkyl ethers may be or include, but are not limited to, one or more of PPG-3 Caprylyl ether, PPG-5 Caprylyl ether, PPG-10 Caprylyl ether, PPG-10 Cetyl ether, PPG-20 Cetyl ether, PPG-28 Cetyl ether, PPG-30 Cetyl ether, PPG-7 Lauryl ether, PPG-10 Lauryl ether, PPG-10 Oleyl ether, PPG-20 Oleyl ether, PPG-23 Oleyl ether, PPG-30 Oleyl ether, PPG-1 1 Stearyl ether, PPG-15 Stearyl ether, or combinations thereof. In a preferred implementation, the personal care composition or the emulsifying system thereof includes at least one polypropylene glycol alkyl ether, more preferably, the polypropylene glycol alkyl ether includes PPG-3 Caprylyl ether. PPG-3 caprylyl ether may be commercially available under the tradename KAO SOFCARE® GP-1 from KAO Chemicals of Tokyo, Japan.

In at least one implementation, the personal care composition may include at least one polypropylene glycol alkyl ether as an emollient. For example, the polypropylene glycol alkyl ether may function as an emollient in the personal care composition. The polypropylene glycol alkyl ether may include PPG-3 Caprylyl ether. PPG-3 caprylyl ether may be commercially available under the tradename KAO SOFCARE® GP-1 from KAO Chemicals of Tokyo, Japan.

The nonionic surfactants may include one or more fatty alcohols or higher alcohols. Illustrative fatty alcohols or higher alcohols may be or include, but are not limited to, straight chain alcohols, such as a C₁₂₋₂₂ fatty alcohol, or preferably a C₁₆₋₁₈ fatty alcohol, lauryl alcohol, myristyl alcohol, cetyl alcohol, behenyl alcohol, stearyl alcohol, cetyl-stearyl alcohol 50/50, or the like, or combinations thereof. Illustrative fatty alcohols may also include branch chain alcohols, such as monostearyl glycerin ether (batyl alcohol), 2-decyltetradecynol, lanolin alcohol, cholesterol, phytosterol, hexyl dodecanol, isostearyl alcohol, octyl dodecanol), or the like, or combinations thereof. In a preferred implementation, the personal care composition or the emulsifying system thereof includes a combination of cetyl alcohol and stearyl alcohol. For example, the emulsifying system may include a combination of cetyl alcohol and stearyl alcohol in a 70/30 blend or mixture.

The amount of any one or more of the surfactants present in the personal care composition or the emulsifying system thereof may vary widely. For example, each of the one or more surfactants may, separately and independently, be present in an amount of from about greater than 0 weight % to less than or equal to about 20 weight %, based on the total weight of the personal care composition or the emulsifying system thereof. For example, each of the one or more surfactants or emulsifiers may, separately and independently, be present in an amount of from about greater than 0 weight %, 0.25 weight %, about 0.5 weight %, about 0.75 weight %, about 1 weight %, about 1.25 weight %, about 1.5 weight %, about 1.75 weight %, about 2 weight %, about 2.25 weight %, about 2.5 weight %, or about 3 weight % to about 3.5 weight %, about 4 weight %, about 4.5 weight %, about 5 weight %, about 5.5 weight %, about 6 weight %, or greater, based on the total weight of the personal care composition or the emulsifying system thereof. In one example, the total amount of the surfactants present in the personal care composition or the emulsifying system thereof may be from greater than 0 wt % to less than 8 wt %, less than 7 wt %, less than 6 wt %, or less than 5 wt %, based on the total weight of the personal care composition or the emulsifying system thereof. In a preferred implementation, the total amount of the surfactants present in the personal care composition or the emulsifying system thereof may be from about 3 wt % to about 7 wt %, more preferably about 4 wt % to about 6 wt %, or about 5 wt %, based on the total weight of the personal care composition or the emulsifying system thereof.

The personal care compositions disclosed herein may include one or more emollients. The one or more emollients may be configured to maintain a soft, smooth, and pliable appearance to the skin and/or hair. The one or more emollients may be or include, but are not limited to, fatty esters, fatty alcohols, or combinations thereof. Illustrative emollients may be or include, but are not limited to, diisopropyl adipate, oleyl alcohol, lanolin, isopropyl myristate, isopropyl palmitate, coco-caprylate, diethylhexyl carbonate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, polyoxypropylene (5) poloxyethylene (20) cetyl ether (PPG-5-Ceteth-20), 2-ethylhexyl isononoate, 2-ethylhexyl stearate, C₁₂ to C₁₆ fatty alcohol, C₁₂ to C₁₆ fatty alcohol lactate, isopropyl lanolate, 2-ethyl-hexyl salicylate, or the like, or combinations thereof. In some implementations, the one or more emollients may be a combination of fatty alcohols. In some implementations, the one or more emollients may be 1-hexadecanol, acetylated lanolin, behenocyl dimethicone, C₁₂₋₁₅ alkyl benzoate, cetearyl octanoate, cocoglycerides, dicaprylate/dicaprate dimethicone copolyol, dimethiconol, dioctyl adipate, glyceryl stearate, isocetyl alcohol, isohexadecane, isopentylcyclohexanone, isopropyl palmitate, lauryllactate, mineral oil, methoxy peg-22/dodecyl glycol copolymer, myristyl lactate, ocryldodecyl neopentanoate, octyl cocoate, octyl palmitate, octyl stearate, octyldodecyl neopentanoate, polyglyceryl-4 isosterate, polyoxyl 40 stearate, polyoxymethylene urea, potassium sorbate, propylene glycol, propylene glycol isoceth-3 acetate, propylene glycol myristyl ether acetate, or combinations thereof. Illustrative emollients may also include, but are not limited to, a high molecular weight saturated and unsaturated fatty alcohol such as, but not limited to, carbitol, lauryl alcohol, myristyl alcohol, cetyl alcohol, isocetyl alcohol, stearyl alcohol, isostearyl alcohol, hydroxystearyl alcohol, oleyl alcohol, ricinoleyl alcohol, behenyl alcohol, erucyl alcohol, 2-octyldodecanyl alcohol, cetearyl alcohol, lanolin alcohol, or the like, or any combination thereof. As discussed above, in at least one implementation, the personal care composition may be free or substantially free of silicone and/or silicone compounds. As such, the personal care composition may be free or substantially free of behenocyl dimethicone, dicaprylate/dicaprate dimethicone copolyol, dimethiconol, or the like. In a preferred implementation, the personal care composition or the emollient thereof may include at least one polypropylene glycol alkyl ether. The polypropylene glycol alkyl ether may include PPG-3 Caprylyl ether. PPG-3 caprylyl ether may be commercially available under the tradename KAO SOFCARE® GP-1 from KAO Chemicals of Tokyo, Japan.

In at least one implementation, the personal care compositions disclosed herein may include one or more emollients in an amount from greater than 0 weight % to less than or equal to 5 weight %, less than or equal to 3 wt %, less than or equal to 1.5 wt %, less than or equal to 1 wt %, less than or equal to 0.8 wt %, less than or equal to 0.5 wt %, or less than or equal to 0.3 wt %, less than or equal to about 0.25 weight %, less than or equal to about 0.1 wt %, based on the total weight of the personal care composition. In a preferred implementation, the one or more emollients is present in an amount of from about 0.1 wt % to about 0.5 wt %, about 0.1 wt % to about 0.3 wt %, or about 0.25 wt %, based on the total weight of the personal care composition.

The personal care composition may include one or more additional optional ingredients. Illustrative optional ingredients may include, but are not limited to, one or more dyes, fragrances, preservatives, thickeners, viscosity modifiers, antioxidants, chelating agents (e.g., EDTA, phosphates, etc.), opacifiers, hydric solvents, hydrotropes, antimicrobials, or the like, or any combination thereof. In at least one implementation, the personal care composition may be free or substantially free of parabens.

All ingredients for use in the compositions described herein should be topically acceptable. As used herein, “topically acceptable” may refer to any ingredient that is present in a composition as described in an amount and form which does not render the composition unsafe for use on surfaces of skin.

The present disclosure may provide methods for preparing any one or more of the personal care compositions disclosed herein. The method may include mixing, combining, stirring, emulsifying, or otherwise contacting two or more of the components/ingredients of the personal care composition disclosed herein with one another. The method may also include heating the components/ingredients before, during, or after mixing, combining, stirring, emulsifying, or otherwise contacting the components with one another.

The present disclosure may include methods for using the personal care composition (e.g., the hair care composition) to deliver conditioning benefits to hair and/or skin of a mammal. The method may include applying to the hair and/or skin of a mammal or human in need thereof any personal care composition including a carrier and a quaternary ammonium compound.

The present disclosure may also include personal care compositions for use in delivering conditioning benefits to hair and/or skin to facilitate wet and/or dry combing, improve softness, improve shine, reduce hair fly-aways, improve hair alignment, facilitate detangling of hair, or combinations thereof.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

An exemplary silicone-free personal care composition having a natural origin index of greater than 95% was evaluated for its efficacy or performance for a plurality of attributes relative to other comparative personal care compositions. Particularly, an exemplary test personal care composition (e.g., hair conditioner) (1) and comparative personal care compositions (2)-(4) were prepared by combining the ingredients or components according to Table 1.

TABLE 1
Personal Care Compositions (1)-(4)

| | | Compar- | Compar- | Compar- |
|---|---|---|---|---|
| | Test (1) | ative (2) | ative (3) | ative (4) |
| Ingredient/Component | (% Wt) | (% Wt) | (% Wt) | (% Wt) |
| Demineralized Water | Q.S. | Q.S. | Q.S. | Q.S. |
| Lactic Acid | 0.60 | — | — | — |
| Sodium Benzoate | 0.21 | — | — | — |
| Potassium Sorbate | 0.06 | — | — | — |
| Brassicamidopropyl | 1.50 | — | — | — |
| dimethylamine | | | | |
| PPG-3 Caprylyl Ether | 0.25 | 0.25 | — | — |
| 70/30 Stearyl/Cetyl | 5.00 | — | 4.00 | 3.70 |
| Alcohol Blend | | | | |
| Fragrance | 0.460 | 0.4 | — | 0.42 |
| Aminosilicone (Silicone- | — | — | 0.4 | — |
| Base Conditioning Agent) | | | | |
| Cetrimonium Chloride | — | — | — | — |
| Mineral Oil | — | — | 0.5 | 0.5 |
| Hydroxycellulose | — | 0.1 | 0.03 | — |
| Benzyl Alcohol/Benzoic | — | 0.3 | 0.3 | 0.3 |
| and Sorbic Acid | | | | |
| DANOX ® HC-30* | — | 5.5 | — | — |
| Cetyl Trimethyl | — | — | 2.5 | 4.58 |
| Ammonium Chloride (29%) | | | | |
| Glyceryl monostearate | — | — | 0.5 | — |
| Caustic Soda (38%) | — | — | — | 0.01 |
| Citric acid | — | — | — | 0.02 |
| Excipients | — | — | 0.7 | 0.03 |
| Total | 100.0 | 100.0 | 100.0 | 100.0 |

*DANOX ® HC-30: Behenamidopropyl Dimethylamine (and) Dipalmitoylethyl Hydroxyethylmonium Methosulfate (and) Cetyl Alcohol (and) Stearyl Alcohol (and) Lactic Acid

As indicated in Table 1, comparative personal care composition (2) was also a silicon-free composition. Comparative personal care compositions (3) and (4) were conventional commercial personal care compositions including a silicone base conditioning agent. The natural origin index of each of the personal care compositions (1)-(4) is summarized in Table 2.

TABLE 2
Natural Origin Index of Personal Care Compositions (1)-(4)

| | Test | Comparative | Comparative | Comparative |
|---|---|---|---|---|
| Ingredient/Component | (1) | (2) | (3) | (4) |
| Total Natural | 98.84 | 98.80 | 91.19 | 94.08 |
| Origin Index | | | | |

The plurality of attributes evaluated included ease of combing wet hair, ease of combing dry hair, softness of dry hair, shine, hair fly-aways, and hair alignment. The compositions (1)-(4) were evaluated by an expert panel and statistical significance was determined via Tukey pairwise comparison. The results are summarized in Table 3.

Hair tresses were obtained from International Hair Importers and Products of Glendale, N.Y. Each of the tresses had a length of about 22 to about 23 cm and was about 3.3 to 3.5 gin weight. Each of the tresses was gently prewashed with 0.5 mL of 20 wt % sodium lauryl ether sulfate (SLES) cleaning solution in triplicate to ensure the hair surface was free of any residual material.

After prewashing, each of the tresses were washed with about 0.5 mL of a low cleansing shampoo for about 1 minute and rinsed for about 1 minute. This was repeated twice. After washing with the low cleansing shampoo, each of the tresses were treated with a respective personal care composition (1)-(4). Specifically, each of the tresses was treated with about 0.5 mL of each of the compositions (1)-(4) for about 1 minute and subsequently rinsed for about 1 minute. Only one application was performed.

For ease of combing, each of the tresses was evaluated by the expert panel immediately after the aforementioned treatment. For dry combing and softness, each of the tresses were air-dried for about 24 hours prior to evaluation.

To evaluate alignment and frizz or hair fly-aways, a Rumba instrument commercially available from Bossa Nova Vision of Los Angeles, Calif. was utilized. Three hair tresses were evaluated for each of the compositions (1)-(4). The Rumba is a polarization imaging system that takes images of a hair sample illuminated with infrared LED and under several polarization states. The instrument takes images of the respective hair tresses, then the image is processed with Rumba software to determine hair orientation using a color index. The comparison of color pixels on each image with the non-reflective background surface is utilized to measure the volume of the hair tress; and thus, evaluate hair orientation, straightness, and alignment.

To evaluate hair shine, a Samba instrument commercially available from Bossa Nova Vision of Los Angeles, Calif. was utilized.

TABLE 3
Performance Efficacy of Personal
Care Compositions (1)-(3) vs. (4)

| | Test | Comparative | Comparative | Comparative |
|---|---|---|---|---|
| Attribute | (1) | (2) | (3) | (4) |
| Wet combing | = | = | = | STD |
| Dry combing | = | = | = | STD |
| Softness | = | = | = | STD |
| Alignment | = | = | = | STD |
| Flyaways | = | = | − | STD |
| Shine | − | − | = | STD |

‘−’ means less than parity
‘=’ means parity

As indicated in Table 3, test and comparative personal care compositions (1)(2) were statistically parity in wet combing, dry combing, softness, and hair alignment. For hair fly-aways, the test and comparative personal care compositions (1)(2) were statistically better than comparative personal care composition (3). As further indicated in Table 3, comparative personal care compositions (3)(4) were statistically better than personal care compositions (1) and (2) with respect to shine.

### Example 2

A monadic blind home use study was conducted to evaluate the test personal care composition (1) of Example 1. To evaluate the test personal care composition, each participant received two products including a shampoo and a conditioner to use for two weeks. Only the hair conditioner, the exemplary personal care composition (1) was evaluated. The monadic blind home study was utilized for evaluating ease of application/distribution in wet hair, ease of finger detangling while rinsing in wet hair, ease of detangling in wet hair, ease of combing in dry hair, hair softness, and ease of combing in dry hair.

Five expert panelists evaluated the head of each of the participants via a half head test. Specifically, the hair on each participant was parted into two equal parts down the middle. Each panelist had 20 final comparisons per each pair of products and each hair type. Hair tresses were evaluated in random pairs. In each paired evaluation, the tress that was evaluated as a better performer received a score of 2 points and the lower performer received one point. The data was analyzed to provide an average and standard deviation. The comparison of treatments was performed applying an ANOVA analysis with α=0.05, and the Tukey method was utilized for grouping and detecting significant differences. The results are summarized in Table 4.

TABLE 4
Overall Performance Efficacy of Personal Care Compositions
(1) and (2) vs (3) of the monadic blind home use test

| | | Test | Compar- | Compar- |
|---|---|---|---|---|
| Condition | Attribute | (1) | ative (2) | ative (3) |
| Wet | Ease to apply & distribute | = | = | STD |
| | Ease to pass finger/hands | = | = | STD |
| | through the hair while rinsing | | | |
| | Ease to untangle | = | − | STD |
| | Ease to comb | = | = | STD |
| Dry | Ease to comb | = | − | STD |
| | Softness | + | = | STD |

‘−’ means less than parity
‘=’ means parity
‘+’ means more than parity

The test personal care composition (1) was at least parity with conventional personal care compositions (3). The test personal care composition (1) was also relatively greater with respect to softness in dry hair. Additionally, test the personal composition (1) exhibited a superior performance in terms of wet hair untangled and softness perception. Also, test personal care composition (1) showed better performance in how manageability, liking appearance, softness on tips, and movement of dry hair (silkiness) in dry evaluation. Furthermore, the personal care composition (1) presented a higher long lasting effect of the fragrance.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

1. A personal care composition, comprising:
an emulsifying system comprising one or more surfactants;
one or more emollients; and
one or more conditioning agents,
wherein the personal care composition comprises a natural origin index of greater than 95%.

2. The personal care composition of claim 1, wherein the personal care composition is substantially free of one or more of silicon, silicon compounds, cetrimonium chloride, propylene glycol, mineral oil, petrolatum, polyethylene glycol, or combinations thereof.

3. The personal care composition of claim 1, wherein the personal care composition is substantially free of silicon and silicon compounds.

4. The personal care composition of claim 1, wherein the personal care composition is substantially free of silicon, silicon compounds, cetrimonium chloride, propylene glycol, mineral oil, petrolatum, and polyethylene glycol.

5. The personal care composition of claim 1, wherein the conditioning agents comprise a cationic conditioning agent.

6. The personal care composition of claim 1, wherein the conditioning agent is present in an amount of from 0.1 weight % to about 5 weight %, based on the total weight of the personal care composition.

7. The personal care composition of claim 1, wherein the one or more surfactants comprise one or more nonionic surfactants.

8. The personal care composition of claim 7, wherein the nonionic surfactants comprise a combination of cetyl alcohol and stearyl alcohol.

9. The personal care composition of claim 1, wherein the one or more surfactants are present in an amount of from greater than 0 weight % to about 20 weight %.

10. The personal care composition of claim 1, wherein the one or more emollients comprises a polypropylene glycol alkyl ether.

11. The personal care composition of claim 1, wherein the emollients are present in an amount of from greater than 0 weight % to about 5 weight %.

12. The personal care composition of claim 1, wherein the natural origin index is greater than 96%.

13. The personal care composition of claim 1, further comprising a preservative.

14. The personal care composition of claim 1, wherein the personal care composition exhibits substantially similar or greater properties as compared to a conventional personal care composition including any one or more of silicone, silicon compounds, cetrimonium chloride, propylene glycol, mineral oil, petrolatum, polyethylene glycol, or combinations thereof.

15. A method for delivering conditioning benefits to hair of a mammal, the method comprising contacting the hair of the mammal in need thereof with the personal care composition of any one of the foregoing claims, wherein the conditioning benefits comprise one or more of facilitating wet combing, facilitating dry combing, softness, shine, reducing hair fly-aways, improving hair alignment, facilitating detangling of hair, or combinations thereof.

16. The personal care composition of claim 5, wherein the cationic conditioning agent comprises brassicamidopropyl dimethylamine.

17. The personal care composition of claim 7, wherein the nonionic surfactants comprise one or more of cetyl alcohol, stearyl alcohol, or combinations thereof.

18. The personal care composition of claim 10, wherein the polypropylene glycol alkyl ether comprises PPG-3 Caprylyl ether.

19. The personal care composition of claim 13, wherein the preservative comprises one or more of sodium benzoate, potassium sorbate, lactic acid, or combinations thereof.

20. The personal care composition of claim 19, wherein the preservative comprises a combination of sodium benzoate, potassium sorbate, and lactic acid.
